(19) United States
(12) Patent Application Publication
(10) Pub. No.: US 2018/0116836 A1
(43) Pub. Date: May 3, 2018

(54) STENT

(71) Applicant: TERUMO KABUSHIKI KAISHA, Tokyo (JP)

(72) Inventor: Ryousuke UEDA, Fuji-shi (JP)

(73) Assignee: TERUMO KABUSHIKI KAISHA, Tokyo (JP)

(21) Appl. No.: 15/785,840

(22) Filed: Oct. 17, 2017

(30) Foreign Application Priority Data
Oct. 28, 2016 (JP) 2016-212324

(51) Int. Cl. A61F 2/915; A61F 2/958

(52) U.S. Cl. CPC A61F 2/915; A61F 2/958; A61F 2230/0069; A61F 2250/001; A61F 2250/0067; A61F 2230/0019; A61F 2002/825; A61F 2002/91583; A61F 2002/91575; A61F 2230/0023; A61F 2250/0026; A61F 2210/0004; A61F 2230/0006; A61F 2230/0026

(57) ABSTRACT

A stent is disclosed that improves passing properties and an expansion force while securing a cross-sectional area of the stent and decreasing a decreasable diameter of the stent. The stent has linear struts, which form an outer periphery of a cylindrical shape and a link portion connecting the struts to each other, in which the struts of the cylindrical shape are mounted on an outer peripheral surface of a dilatable and deformable balloon. The stent has a plurality of strut cross-sectional portions in a cross section perpendicular to an axial direction of the struts of the cylindrical shape, the strut cross-sectional portions adjacent to each other in a circumferential direction of the struts have shapes different from each other, and surfaces of the strut cross-sectional portions adjacent to each other are in contact with each other when the struts are mounted on the outer peripheral surface of the balloon.

### CROSS-REFERENCES TO RELATED APPLICATIONS

This application claims priority to Japanese Application No. 2016-212324 filed on Oct. 28, 2016, the entire content of which is incorporated herein by reference.

### TECHNICAL FIELD

The present disclosure relates to a stent used for treating, for example, a stenosed site or a closed site caused in a body lumen such as a blood vessel.

### BACKGROUND DISCUSSION

A stent is indwelled in a stenosed site or a closed site caused in a body lumen such as a blood vessel in an expanded state and maintains an open state of the body lumen, and strength for maintaining the expanded state is required. A stent mounted in a stent delivery system can have a high sliding resistance and hardly bends. Therefore, passing properties when the stent is made to pass through a bent portion or a stenosed site of a blood vessel are particularly required.

The stent delivery system which delivers a stent to a body lumen includes an elongated shaft portion, a balloon provided on a distal side of the shaft portion, and the stent mounted on an outer peripheral surface of the balloon, which is in a deflated state, in a state where a diameter of the stent decreases due to compressive deformation. The stent delivery system delivers the stent to a stenosed site or a closed site of a body lumen. Thereafter, the stent in these lesion areas enters a state where the diameter of the stent increases due to dilation of the balloon and is indwelled in these lesion areas in a state where the diameter of the stent increases due to deflation of the balloon.

However, in the stent of the stent delivery system, the stent is mount (crimp) on the outer peripheral surface of the balloon in a state where the diameter of the stent decreases as much as possible in order to improve passing properties of the stent. By doing this, it is possible to suppress an increase in an outer diameter (profile) of a shaft portion on the distal side when the stent is mounted on the outer peripheral surface of the balloon and to improve the passing properties of the stent. For example, in a stent in JP-A-2015-177956, a cross-sectional shape of a skeleton constituting the stent is formed such that a width dimension increases from an inner peripheral surface toward the outer peripheral surface due to the change in a thickness direction of the cross-sectional shape. As a result, the passing properties of the stent are improved by decreasing a decreasable diameter of the stent.

However, when the cross-sectional shape perpendicular to an axial direction of the stent is formed such that a width dimension increases from the inner peripheral surface toward an outer peripheral surface, the stent is mounted on the balloon in a state where the inner peripheral surface of the stent of which the width dimension is small is in contact with the outer peripheral surface of the balloon. As a result, there is a concern that the inner peripheral surface of the stent of which the width dimension is small may damage the balloon. In addition, since the contact area of the inner peripheral surface of the stent with respect to the outer surface of the balloon decreases, there is a concern that it may not be possible to reliably transmit a dilation force of the balloon to the stent and to sufficiently expand the stent. Furthermore, in the cross section perpendicular to an axial direction of the stent, the cross-sectional area of the stent formed such that the width dimension of the cross-sectional shape increases from the inner peripheral surface toward the outer peripheral surface is smaller than that of the stent of which the cross-sectional shape is rectangular. Therefore, there is a concern that an expansion force of the stent for keeping a lesion area in an open state may decrease. As described above, there is a concern that the technique of decreasing the decreasable diameter of the stent may cause an unprepared decrease in the cross-sectional area of the stent or a failure of expansion of the stent when the width dimension of the cross section of the stent is simply changed in the thickness direction in the cross section perpendicular to the axial direction of the stent.

### SUMMARY

A stent is disclosed that improves passing properties and an expansion force while securing a cross-sectional area of the stent and decreasing a decreasable diameter of the stent.

In accordance with an exemplary embodiment, a stent is disclosed, which has a plurality of linear struts, which form an outer periphery of a cylindrical shape in which a gap is formed, and a link portion connecting the struts to each other in the gap, in which the struts of the cylindrical shape are mounted on an outer peripheral surface of a dilatable and deformable balloon in a state where the diameter of the struts decreases, in which when the struts are mounted on the outer peripheral surface of the balloon which is in a deflated state, in a state where the diameter of the struts decreases, the stent has a plurality of strut cross-sectional portions in a cross section perpendicular to an axial direction of the struts of the cylindrical shape, the strut cross-sectional portions adjacent to each other in a circumferential direction of the struts of the cylindrical shape have shapes different from each other, and surfaces of the strut cross-sectional portions adjacent to each other in the circumferential direction are in contact with each other when the struts are mounted on the outer peripheral surface of the balloon, which is in the deflated state, in a state where the diameter of the struts decreases.

According to the stent having the above-described configuration, when the struts are mounted on the outer peripheral surface of the balloon which is in a deflated state, in a state where the diameter of the struts decreases, the stent has a plurality of strut cross-sectional portions in a cross section perpendicular to an axial direction of the struts of the cylindrical shape, the strut cross-sectional portions adjacent to each other in a circumferential direction of the struts of the cylindrical shape have shapes different from each other, and surfaces of the strut cross-sectional portions adjacent to each other in the circumferential direction are in contact with each other when the struts are mounted on the outer peripheral surface of the balloon, which is in the deflated state, in the state where the diameter of the struts decreases. For this reason, it is possible to decrease a decreasable diameter of the stent while sufficiently securing the cross-sectional area of the stent when the stent is mounted on the outer surface of the balloon, compared to a stent in the related art in which the cross-sectional shape of struts is rectangular or a stent in the related art in which the width dimension of struts increases from an inner peripheral surface side toward an outer peripheral surface side of the stent. As a result, it is possible to improve passing properties and expansibility of the stent.

In accordance with an exemplary embodiment, a stent is disclosed comprising: a plurality of linear struts, which form an outer periphery of a cylindrical shape; and a link portion connecting the plurality of linear struts to each other, wherein the stent has a plurality of strut cross-sectional portions in a cross section perpendicular to an axial direction of the plurality of linear struts of the cylindrical shape, and wherein the plurality of strut cross-sectional portions adjacent to each other in a circumferential direction of the plurality of linear struts of the cylindrical shape have shapes different from each other.

In accordance with an exemplary embodiment, a method is disclosed of decreasing an outer diameter of a stent in a deflated state, the method comprising: forming an outer periphery of a cylindrical shape with a plurality of linear struts having a gap between the plurality of linear struts; connecting the plurality of linear struts to each other in the gap with a link portion; and mounting the plurality of linear struts of the cylindrical shape on an outer peripheral surface of a dilatable and deformable balloon, wherein the stent has a plurality of strut cross-sectional portions in a cross section perpendicular to an axial direction of the plurality of linear struts of the cylindrical shape, the wherein the plurality of strut cross-sectional portions adjacent to each other in a circumferential direction of the plurality of linear struts of the cylindrical shape have shapes different from each other, and surfaces of the strut cross-sectional portions are in contact with each other when the plurality of linear struts are mounted on the outer peripheral surface of the balloon.

### BRIEF DESCRIPTION OF THE DRAWINGS

FIG. 1 is a perspective view of a stent of an embodiment in a state (natural state) where a diameter of the stent is expanded.

FIG. 2 is a developed view in which a part of the outer periphery of the stent of the embodiment is developed by being linearly cut along an axial direction.

FIG. 3A is an enlarged perspective view of a cross section perpendicular to the axial direction in a portion A of FIG. 2 when the stent of the embodiment is in a state where the diameter of the stent decreases.

FIG. 3B is a cross-sectional view, which is taken along line IIIB-IIIB of FIG. 1 when the stent of the embodiment is in a state where the diameter of the stent decreases, and is perpendicular to the axial direction.

FIGS. 4A and 4B are cross-sectional views taken along line IV-IV of FIG. 3A.

FIG. 5A is an enlarged view of a portion A of FIG. 2.

FIG. 5B is a cross-sectional view of a bent portion in a direction perpendicular to the axial direction, which is taken along line VB-VB of FIG. 5A.

FIG. 5C is a cross-sectional view corresponding to FIG. 5B showing how the bent portion is curved when the stent enters a state where the diameter of the stent decreases.

FIGS. 6A and 6B are views for illustrating an action and an effect of the stent of the embodiment, wherein FIG. 6A is a cross-sectional view perpendicular to an axial direction of a cylindrical shape of a stent of a comparative example in a state where the diameter of the stent decreases and FIG. 6B is a view which is obtained by superimposing the cross-sectional view perpendicular to the axial direction of the cylindrical shape of the stent of the comparative example in a state where the diameter of the stent decreases on the cross-sectional view perpendicular to the axial direction of the cylindrical shape of the stent of the embodiment in a state where the diameter of the stent decreases, and corresponds to a view obtained by superimposing FIG. 6A on the drawing of FIG. 3B.

FIGS. 7A, 7B, and 7C are cross-sectional views perpendicular to an axial direction and correspond to cross-sectional views taken along line IV-IV of FIG. 3A of stents (in a width direction of a straight portion) according to Modification Example 1, Modification Example 2, and Modification Example 3, respectively.

### DETAILED DESCRIPTION

Hereinafter, an embodiment of the present disclosure will be described with reference to the accompanying drawings. Note that dimensional ratios in the drawings are exaggerated and are different from the actual ratios for the convenience of description.

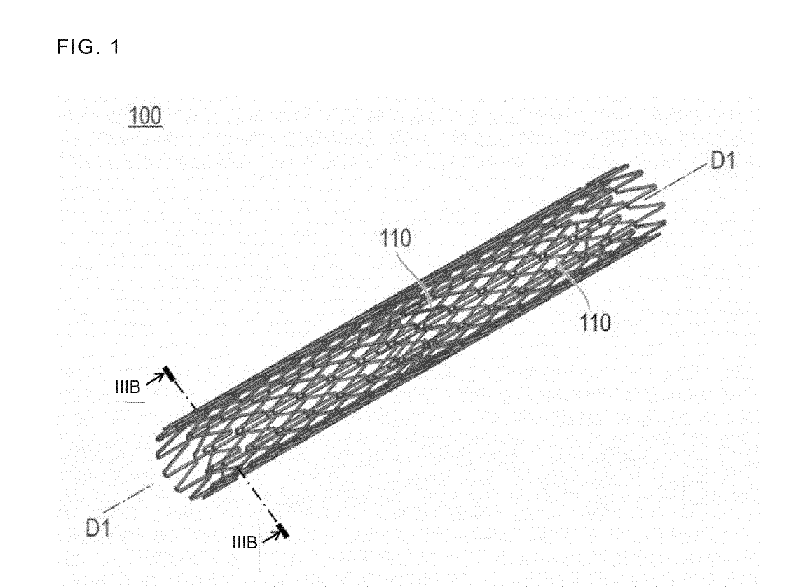

FIGS. 1 to 5C are schematic views showing a structure of a stent 100 of the embodiment. Hereinafter, the stent 100 of the embodiment will be described with reference to FIGS. 1 to 5C.

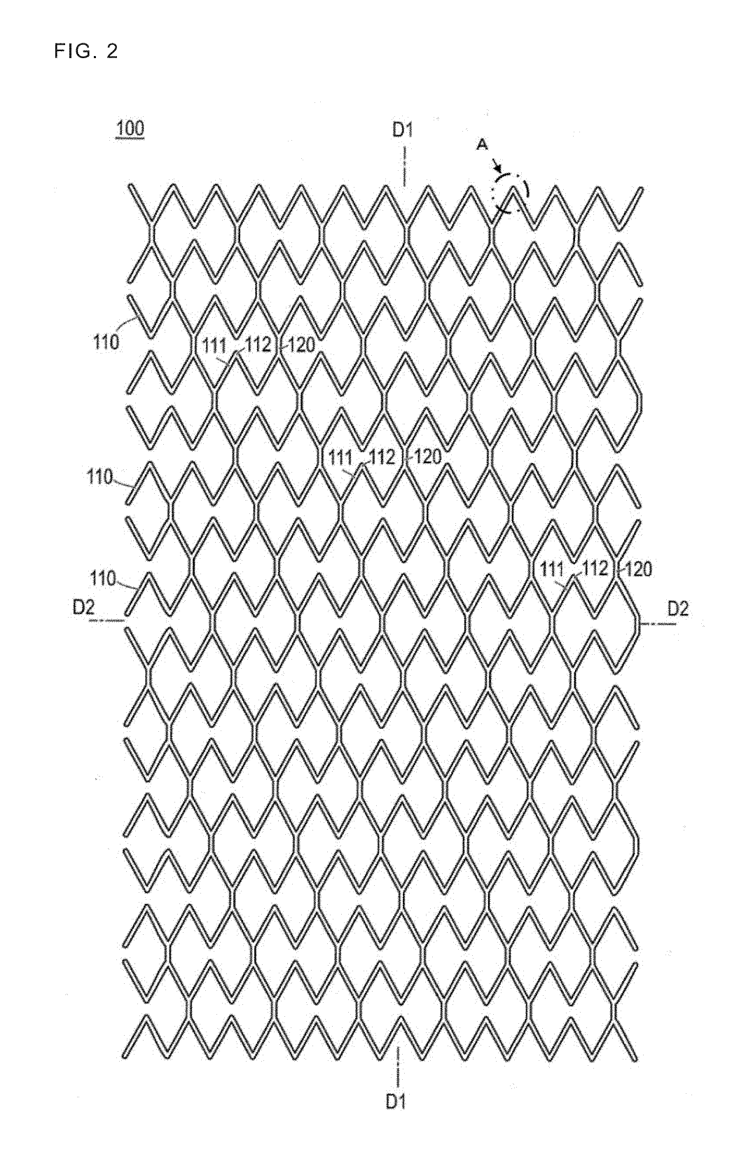

As shown in FIGS. 1 and 2, the stent 100 of the embodiment has a plurality of struts 110, which are linear components, and a plurality of link portions 120 connecting the plurality of struts 110 to each other.

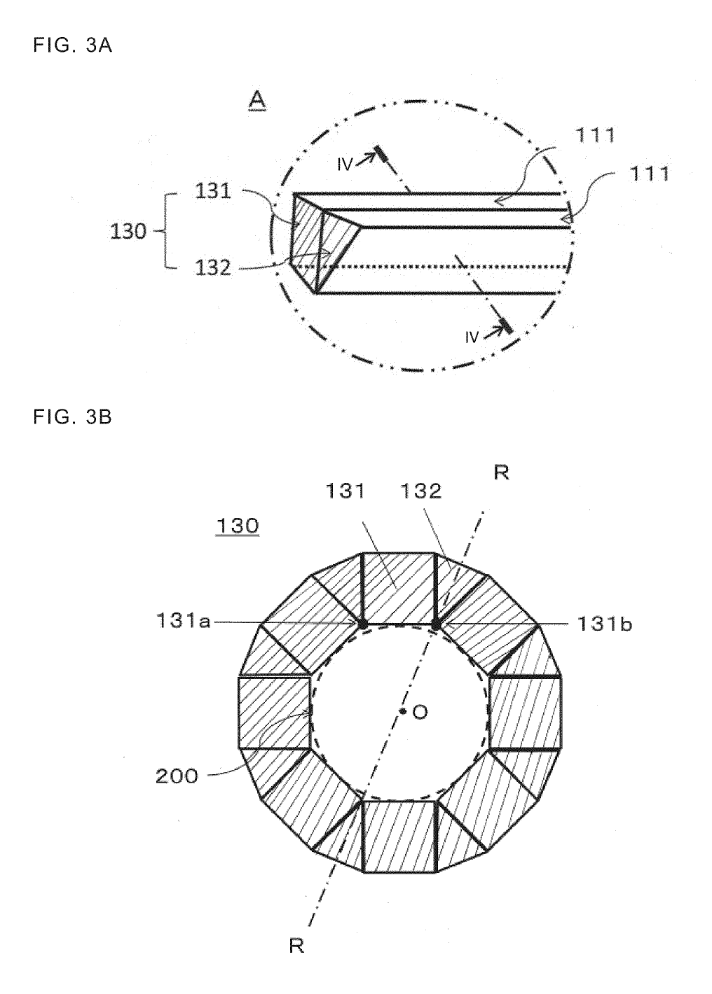

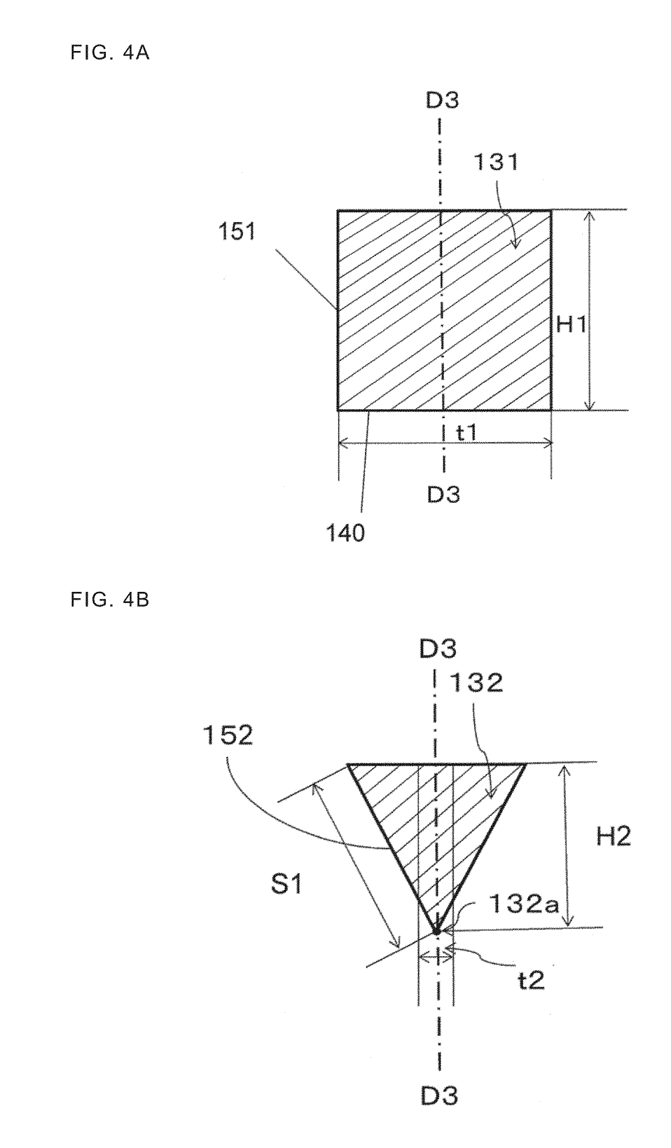

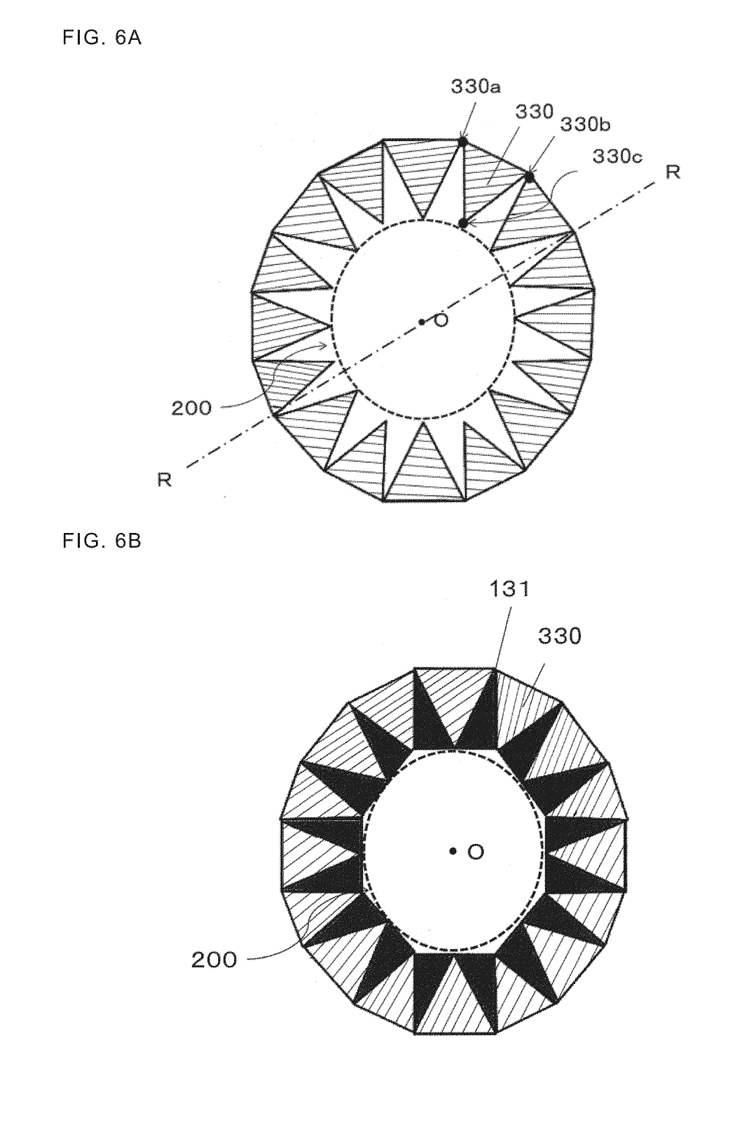

Note that, in the specification, an axial direction of a cylindrical shape formed using the struts 110 is simply denoted as an “axial direction D1” (refer to FIGS. 1 and 2), a circumferential direction of the cylindrical shape is simply denoted as a “circumferential direction D2” (refer to FIG. 2), a radial direction of the cylindrical shape is simply denoted as a “radial direction R” (refer to FIGS. 3B and 6A), a thickness direction of the struts 110 is simply denoted as a “thickness direction D3” (refer to FIGS. 4A and 4B). The width direction of the struts 110 is simply denoted as a “width direction W”.

The struts 110 form an outer periphery of the cylindrical shape in which a gap is formed, as shown in FIG. 1. As shown in FIG. 2, the struts 110 have a plurality of straight portions 111 and a plurality of bent portions 112 interlocking the straight portions 111 extending in directions different from each other. The struts 110 extend in the circumferential direction D2 while being folded back in a wave shape to form an endless tubular shape.

The plurality of the struts 110 are provided along the axial direction D1 as shown in FIG. 2. The plurality of struts 110 provided along the axial direction D1 are connected to each other through the link portions 120.

The link portions 120 connects the struts 110 to each other in a gap between the struts 110 adjacent along the axial direction D1 as shown in FIG. 2.

The link portions 120 according to the present embodiment are provided along the axial direction D1. Note that the link portions 120 may be provided along a direction intersecting with the axial direction D1.

The plurality of link portions 120 is disposed at predetermined intervals in the circumferential direction D2. Note that the location where the link portions 120 are disposed is not limited to the location shown in FIG. 2 and can be appropriately changed as long as the plurality of struts 110 provided along the axial direction D1 are connected to each other.

In the stent 100, the struts 110 and the link portions 120 are integrally formed.

The material forming the stent 100 is, for example, a biodegradable material which is degraded in vivo. Examples of such a material include biodegradable synthetic polymeric materials such as polylactic acid, polyglycolic acid, lactic acid-glycolic acid copolymers, polycaprolactone, lactic acid-caprolactone copolymers, glycolic acid-caprolactone copolymers, and poly-y-glutamic acid, biodegradable natural polymer materials such as collagen, biodegradable metallic materials such as magnesium and zinc. Note that the material forming the stent 100 is not limited to the biodegradable material, and the stent may be formed, for example, of a non-biodegradable material which is not degraded in vivo. Examples of such a material include stainless steel, a cobalt-based alloy such as a cobalt-chromium alloy (for example, a CoCrWNi alloy), elastic metal such as a platinum-chromium alloy (for example, a PtFeCrNi alloy), and a super-elastic alloy such as a nickel-titanium alloy.

In addition, the stent 100 may include a coating body (not shown in the drawing) containing a medicine on its surface. In accordance with an exemplary embodiment, the coating body is preferably formed on an outer surface side of the stent 100, which is to be come into contact with the blood vessel wall, but the present disclosure is not limited thereto.

The coating body contains a medicine capable of suppressing proliferation of neointima, and a medicine carrier for carrying the medicine. Note that the coating body may be formed of only the medicine. The medicine contained in the coating body is at least one selected from the group consisting, for example, of sirolimus, everolimus, zotarolimus, and paclitaxel. The constituent material of the medicine carrier is not particularly limited, but a biodegradable material is preferable, and the same material as that of the stent 100 is applicable.

Next, a cross-sectional shape of the stent 100 will be described with reference to FIGS. 3A and 3B. FIG. 3A is an enlarged perspective view of a cross section perpendicular to the axial direction D1 in a portion A of FIG. 2 when the stent 100 is in a state where a diameter of the stent decreases. FIG. 3B is a cross-sectional view which is taken along line IIIB-IIIB of FIG. 1 when the stent 100 is in a state where the diameter of the stent decreases, and is perpendicular to the axial direction D1.

When the stent 100 is mounted on an outer surface of the balloon 200 in a state where the diameter of the stent decreases and the stent is deformed, the stent has a plurality of strut cross-sectional portions 130 formed of different cross-sectional shapes of the struts 111 which are adjacent to each other in the circumferential direction, in a cross section perpendicular to the axial direction D1 of the struts 110 of the cylindrical shape. When the stent 100 is mounted on the outer surface of the balloon 200 in a state where the diameter of the stent decreases and the stent is deformed, the strut cross-sectional portions 130 are in close proximity to each other in the circumferential direction D2 and the side surfaces of the strut cross-sectional portions 130 facing each other in the circumferential direction D2 are in contact with each other, thereby decreasing the decreasable diameter of the stent 100. In addition, each of the plurality of stent cross-sectional portions 130 provided in the stent 100 is formed of a strut cross-sectional portion 130 for securing the cross-sectional area of the stent 100 and a strut cross-sectional portion 130 for decreasing the decreasable diameter of the stent 100.

The decreasable diameter referred to herein is a diameter of a circle passing through the apex of each of the struts 111 on the outer peripheral surface of the stent 100, which is in a state where the diameter of the stent decreases, and defines a diameter decrease limit amount of the stent 100. That is, the decreasable diameter of the stent 100 changes in accordance with the number of and the shape of struts 111 forming the outer peripheral surface or an inner peripheral surface of the stent 100 which is in a state where the diameter of the stent decreases. Note that the balloon 200 shown by a dotted line in FIG. 3B is in a deflated state.

In accordance with an exemplary embodiment, it can be preferable that the strut cross-sectional portions 130 are provided on the cross section in the direction perpendicular to the axial direction D1 of the struts 110 at both end portions of the stent 100. The struts 110 constituting both end portions of the stent 100 can have a dense structure in order to secure a compressive force of the stent 100 with respect to the body lumen wall. As a result, the decreasable diameter at both end portions of the stent 100 tends to become particularly large. Furthermore, in a case where the decreasable diameter at both end portions of the stent 100 increases, the level difference (clearance) caused by the difference in an outer diameter at both end portions of the stent 100 and the balloon 200 when the stent 100 is mounted on the outer surface of the balloon 200 increases. For this reason, the decreasable diameter of the stent 100 at both end portions is decreased by providing the plurality of strut cross-sectional portions 130 on the cross section of the struts 111 at both end portions of the stent 100, and therefore, the clearance between both end portions of the balloon 200 and the stent 100 can be suppressed.

As shown in FIG. 3A, the plurality of strut cross-sectional portions 130 are adjacent to each other along the circumferential direction D2 and include a strut 111 and the other strut 111 which are formed of different cross-sectional shapes adjacent to each other along the circumferential direction D2.

The plurality of strut cross-sectional portions 130 are formed of a first cross section 131 provided in the one strut 111 and a second cross section 132 provided in the other strut 111. The plurality of strut cross-sectional portions 130 are formed by alternately arranging the first cross section 131 and the second cross section 132 adjacent to each other along the circumferential direction D2 as shown in FIG. 3B. With such a configuration, it is possible to uniformly decrease the diameter of each of the struts 111 constituting the plurality of strut cross-sectional portions 130, and therefore, it is possible to further decrease the decreasable diameter of the stent 100. As will be described in detail below, in the stent 100, the first cross section 131 is provided to decrease the decreasable diameter of the stent 100 and the second cross section 132 is provided to secure the cross-sectional area of the stent 100.

In the present exemplary embodiment, the first cross section 131 has a rectangular shape and the second cross section 132 has an inverted triangular shape. In addition, the first cross section 131 and the second cross section 132 are preferably formed such that profiles of the respective cross sections are in an offset relation in the thickness direction. With such a configuration, in a state where the diameter of the stent 100, which are mounted on the outer surface of the balloon 200, decreases, it is possible to make the first cross section 131 and the second cross section 132 be in closer proximity to each other and to make the side surfaces of the first cross section 131 and the second cross section 132 facing each other in the circumferential direction D2 come into contact with each other within a wider range. Therefore, the decreasable diameter of the stent 100 can be further decreased.

The first cross section 131 forms the inner peripheral surface of the stent 100 abutting on the outer surface of the balloon 200 when the stent 100 is in the state where the diameter of the stent decreases. At this time, the decreasable diameter of the stent 100 is defined by the first cross section 131.

Eight first cross sections 131 are provided along the circumferential direction D2, and apexes 131a and 131b on the inner peripheral surface side of a first cross section 131 are respectively in contact with the apexes 131b and 131a of the other first cross section 131 adjacent to each other in the circumferential direction D2. With such a configuration, as will be described in detail below, it is possible to decrease the area where the second cross section 132 abuts on the outer peripheral surface of the balloon 200, and therefore, it is possible to suitably prevent any damage caused on the balloon 200 by the second cross section 132.

The number of struts 111 having the first cross sections 131 constituting the plurality of stent cross-sectional portions 130 is not particularly limited, but is desirably half the number of struts constituting a cross section of a stent in the related art. That is, when the number of struts 111 having the first cross sections 131 constituting the plurality of stent cross-sectional portions 130 of the present embodiment is eight (8) as shown in FIG. 3B, the number of the same struts constituting a cross section of a stent in the related art becomes sixteen (16). With such a configuration, it is possible to decrease the number of struts 111 defining the decreasable diameter of the stent 100, and therefore, it is possible to decrease the decreasable diameter of the stent 100.

As shown in FIGS. 4A and 4B, the length t1 of the width direction W of the inner peripheral surface of the first cross section 131 is preferably formed to be longer than the length t2 of the width direction W of the inner peripheral surface of the second cross section 132. With such a configuration, it is possible to make the area of the first cross section 131 abutting on the outer surface of the balloon 200 is larger than that of the second cross section 132, and therefore, it is possible to reliably transmit a dilation force of the balloon 200 to the first cross section 131.

As shown in FIGS. 4A and 4B, the length H1 of the first cross section 131 in the thickness direction D3 is preferably set to be the same as the length S1 of the side surface 152 of the second cross section 132 which faces the first cross section 131 in the circumferential direction D2. With such a configuration, it is possible to suppress the level difference caused on the outer peripheral surfaces of the plurality of strut cross-sectional portions 130 due to protrusion of any of the first cross section 131 and the second cross section 132 in the radial direction R in the stent 100 in the state where the diameter of the stent decreases, and therefore, it is possible to improve the passing properties of the stent 100 in the body lumen.

In addition, a guiding portion (not shown in the drawing) that guides expansion of the second cross section 132 by transmitting a dilation force of the balloon 200 to adjacent second cross section 132 during the dilation of the balloon 200 may be provided on a side surface 151 facing the second cross section 132 in the circumferential direction D2 of the first cross section 131. The guiding portion is not particularly limited as long as a friction coefficient is set to be high, and examples thereof include a fine uneven structure provided on the outer surface of the struts 111. Note that the guiding portion may be provided on only the first cross section 131 and may be provided on both of the first cross section 131 and the second cross section 132.

As shown in FIG. 3B, eight second cross sections 132 are disposed so as to be accommodated in grooves formed between the eight first cross sections 131 arranged along the circumferential direction D2 in the plurality of strut cross-sectional portions 130 of the stent 100 in the state where the diameter of the stent decreases. Note that the shape of a groove is not particularly limited, but a shape along the outer shape of a second cross section 132 may be used.

As shown in FIG. 3B, the grooves in which the second cross sections 132 are disposed are formed to have a shape tapered toward a center O inward in the radial direction R. For this reason, when the stent 100 enters the state where the diameter of the stent decreases, the second cross sections 132 are accommodated toward the center O inward in the radial direction R along the shapes of the grooves. At this time, the apexes 132a positioned on the inner peripheral surfaces of the second cross sections 132 abut on contact points of the apexes 131a and 131b of the plurality of first cross sections 131 arranged along the circumferential direction D2. With such a configuration, in accordance with an exemplary embodiment, it can be preferable to prevent the apexes 132a of the second cross sections 132 from abutting on the outer surface of the balloon 200 and to prevent any damage on the balloon 200. Furthermore, positional displacement in the circumferential direction D2 of the second cross sections 132 during the dilation of the balloon 200 can be prevented.

In addition, the second cross sections 132 are preferably set not to form the inner peripheral surface of the stent 100 which is in the state where the diameter of the stent decreases. That is, it is possible to decrease the decreasable diameter of the stent 100 by forming the inner peripheral surface of the plurality of strut cross-sectional portions 130 using only the first cross sections 131. In accordance with an exemplary embodiment, it can be desirable that the second cross sections 132 function to secure the expansion force of the stent 100 when the stent 100 enters a state where the diameter of the stent increases.

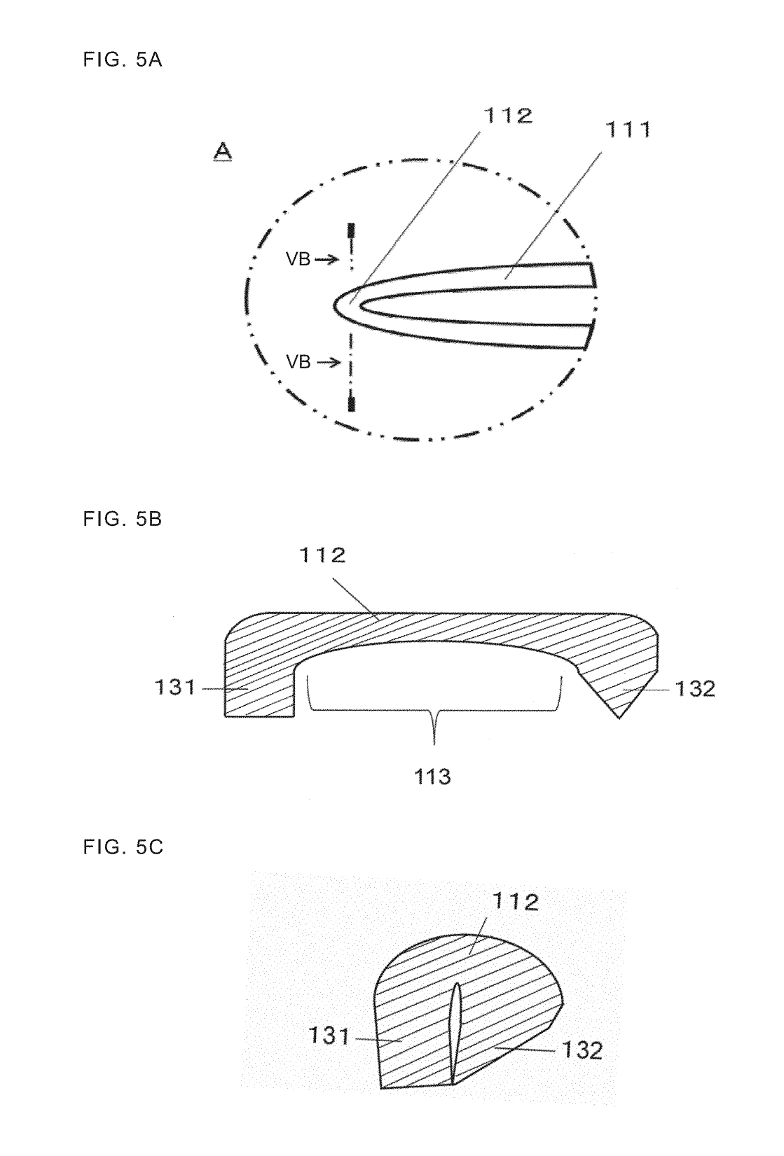

As shown in FIG. 5B, the bent portion 112 interlocking the struts 111 extending in directions different from each other has a hollow 113 formed from the inner peripheral side toward the outer peripheral side of the struts 110 at a central portion of a cross section (a cross section perpendicular to the axial direction D1) taken long line VB-VB of FIG. 5A. With such a configuration, as shown in FIG. 5C, the first cross section 131 and the second cross section 132 adjacent to each other in the circumferential direction D2 in the bent portion 112 are curved in a direction where the first cross section and the second cross section are in close proximity to each other when the stent 100 enters the state where the diameter of the stent decreases. Therefore, it is possible to more suitably decrease the decreasable diameter of the stent 100. In addition, when the stent 100 is in the state where the diameter of the stent increases, the bent portion 112 tends to be expanded outward in the radial direction R, and therefore, it is possible to easily make the bent portion 112 contacted with the body lumen wall.

In accordance with an exemplary embodiment, the first cross sections 131 and the second cross sections 132 constituting such a plurality of strut cross-sectional portions 130 can be formed by appropriately applying an etching method in which masking called photofabrication and chemical agents are used, an electric discharge machining method using a mold, a cutting method, and the like thereto. The cutting method is, for example, mechanical polishing, or laser cutting. Thereafter, finishing processing such as chemical polishing or electrolytic polishing, or heat treatment such as annealing is appropriately performed.

Next, the action and the effect of the stent 100 of the present embodiment will be described.

The stent 100 is delivered, for example, to a stenosed site or a closed site caused in a body lumen such as blood vessels, bile ducts, the trachea, the esophagus, or the urethra using a medical equipment for stent delivery such as balloon catheter. When delivering the stent 100, the stent 100 is mounted on the outer peripheral surface of the balloon 200 in a state where the diameter of the stent decreases.

The delivered stent 100 enters a state where the diameter of the stent increases in accordance with dilation of the balloon 200 in a stenosed site or a closed site within a blood vessel. Note that the stent 100 may be a self-expanding type.

When the stent 100 of the present embodiment is mounted on the outer peripheral surface of the balloon 200 in a state where the diameter of the stent decreases, the stent has a plurality of strut cross-sectional portions 130, which have different cross-sectional shapes of the struts 111 adjacent to each other in the circumferential direction D2, in a cross section perpendicular to the axial direction D1 of the struts 110 of the cylindrical shape. In accordance with an exemplary embodiment, the plurality of strut cross-sectional portions 130, the side surfaces of the strut cross-sectional portions 130 facing each other in the circumferential direction D2 can be brought into contact with each other since the strut cross-sectional portions 130 adjacent to each other in the circumferential direction D2 are in close proximity to each other when the diameter of the stent 100 decreases and the stent is deformed to be mounted on the outer surface of the balloon 200. Therefore, the decreasable diameter of the stent 100 decreases. Accordingly, when delivering the stent 100 to the body lumen, it is possible to deliver the stent 100 thereto in a state where the decreasable diameter of the stent 100 is made to be small. Therefore, the passing properties of the stent 100 can be improved.

In addition, the stent 100 has the plurality of strut cross-sectional portions 130 at both end portions of the stent 100. Accordingly, the stent 100 decreases the decreasable diameter at both end portions, and the clearance caused by the difference in the outer diameter at both end portions of the stent 100 and the balloon 200 during the delivery of the stent 100 can be suppressed. For this reason, it is possible to prevent the shape of the stent 100 from being unintentionally changed or the stent 100 from dropping out due to the stent 100 being caught in a bent site, a stenosed site, or the like during the delivery of the stent 100. Therefore, the passing properties of the stent 100 can be further improved.

Furthermore, in the stent 100, it is possible to sufficiently suppress increase in the decreasable diameter of the stent 100 at both end portions by providing the plurality of stent cross-sectional portions 130 at both end portions of the stent 100 even if the struts 110 of both end portions of the stent 100 are made to have a dense structure in order to increase a compression force of the stent 100, which has entered the state where the diameter of the stent increases, with respect to the body lumen wall.

In accordance with an exemplary embodiment, the plurality of strut cross-sectional portions 130 of the stent 100 include strut cross-sectional portions 130 that function to form the inner peripheral surface of the stent 100 in the state where the diameter of the stent decreases, in order to decrease the decreasable diameter of the stent 100. Furthermore, the plurality of strut cross-sectional portions 130 include strut cross-sectional portions 130 that function to maintain an expansion force of the stent 100 for supporting a lesion area when the stent 100 is made to enter the state where the diameter of the stent increases. That is, since the plurality of strut cross-sectional portions 130 of the stent 100 have two types of the strut cross-sectional portions 130 for decreasing the decreasable diameter and the strut cross-sectional portions 130 for maintaining the expansion force, the passing properties and the expansion force of the stent 100 can be improved.

Hereinafter, the plurality of strut cross-sectional portions 130 of the stent 100 of the present embodiment and a plurality of strut cross-sections 330 of a stent 300 according to a comparative example will be compared with each other with reference to FIGS. 6A and 6B.

First, the plurality of strut cross-sectional portions 330 formed on a cross section perpendicular to the axial direction D1 when the stent 300 of the comparative example is mounted on the outer surface of the balloon 200 in a state where the diameter of the stent 300 decreases will be described with reference to FIG. 6A. Note that the same structure as that in the present embodiment will be described below using the same reference numerals.

The cross-sectional shape of the plurality of strut cross-sectional portions 330 of the comparative example is formed of only an inverted triangular shape, and the plurality of strut cross-sectional portions 330 are disposed so as to be adjacent to each other along the circumferential direction D2. Note that the number of struts 111 constituting the plurality of strut cross-sectional portions 330 is 16. The strut cross-sectional portions 330 are formed such that the length of the width direction W along the thickness direction D3 is long. For this reason, contact between the strut cross-sectional portions 330 adjacent to each other along the circumferential direction D2 and apexes 331c on an inner peripheral surface side of the strut cross-sectional portions 330 is suppressed when decreasing the diameter of the stent 300. As a result, the decreasable diameter of the stent 300 decreases. In the plurality of strut cross-sectional portions 330, it is possible to decrease the diameter until apexes 331a and 331b of one strut cross-sectional portion 330 positioned on the outer peripheral surface side of the stent 300 between the strut cross-sectional portions 330 adjacent to each other along the circumferential direction D2 come into contact with apexes 331b and 331a of the other adjacent strut cross-sectional portions 330. That is, the decreasable diameter of the stent 300 of the comparative example is defined by all 16 strut cross-sectional portions 330. In such a stent 300 of the comparative example, it is possible to further decrease the decreasable diameter compared to a stent in the related art which is formed to have 16 struts 111, which are the same number as that of the plurality of strut cross-sectional portions 330, and in which the cross-sectional shapes of the struts 111 are only rectangular.

However, since the cross-sectional shape of the plurality of strut cross-sectional portions 330 of the comparative example is formed of only the inverted triangular shape in order to decrease the decreasable diameter of the stent 300, the whole area (total area) of the plurality of strut cross-sectional portions 330 decreases. For this reason, when the diameter of the stent 300 increases due to dilation of the balloon 200, there is a concern that a force supporting a lesion area using the stent 300 may decrease. Furthermore, the apexes 331c of the plurality of strut cross-sectional portions 330 abutting on the outer surface of the balloon 200 have a sharp tip. Therefore, there is a concern that the balloon 200 may be damaged or a concern that a failure of expanding the stent 300 may be caused since a dilation force of the balloon 200 is not sufficiently transmitted to the struts 111 due to a decreased contact area between the balloon 200 and the struts 111.

FIG. 6B is a view obtained by superimposing the plurality of strut cross-sectional portions 330 of the stent 300 of the comparative example on the plurality of strut cross-sectional portions 130 of the stent 100 of the present embodiment. In FIG. 6B, the structure of the present embodiment is shown in black in order to clarify the difference between the present embodiment and the comparative example.

As shown in FIG. 6B, the plurality of strut cross-sectional portions 130 of the stent 100 of the present embodiment include two types of the first cross section 131 and the second cross section 132 unlike the plurality of strut cross-sectional portions 330 of the stent 300 of the comparative example. In accordance with an exemplary embodiment, the first cross section 131 and the second cross section 132 are arranged so as to be adjacent to each other along the circumferential direction D2. The first cross section 131 has a rectangular shape and the second cross section 132 has an inverted triangular shape similarly to the comparative example. Note that, in FIG. 6B, the second cross sections 132 of the present embodiment having the same structure as that of the strut cross-sectionals 330 of the comparative example are covered by the plurality of strut cross-sectional portions 330 of the comparative example. Therefore, only a part of the first cross sections 131 is shown in black.

When the stent 100 enters a state, in which the diameter of the stent decreases, and is mounted on the outer peripheral surface of the balloon 200, the inner peripheral surface of the stent 100 is formed of only the first cross sections 131. That is, the decreasable diameter of the stent 100 is defined by the first cross sections 131. In such a stent 100 of the present embodiment, it is possible to form the inner peripheral surface of the stent 100 with half the number of struts in the state where the diameter of the stent decreases, and therefore, it is possible to further decrease the decreasable diameter compared to the stent in the related art which is formed to have the same number of struts 111 as that of the plurality of strut cross-sectional portions 130 of the stent 100, and in which the cross-sectional shape of the struts 111 is only rectangular.

In addition, since the first cross sections 131 having a longer lengths of the width direction W compared to those of the second cross sections 132 form the inner peripheral surface of the stent 100 in the state where the diameter of the stent decreases, it is possible to secure a sufficient contact area of the struts 111 with respect to the outer surface of the balloon 200 in the stent 100. For this reason, a dilation force of the balloon 200 to the first cross sections 131 can be reliably transmitted unlike the stent 300 of the comparative example, and therefore, a failure of expansion of the stent 100 can be prevented.

The second cross sections 132 are disposed so as to be accommodated in grooves formed between the eight first cross sections 131 arranged along the circumferential direction D2 in the plurality of strut cross-sectional portions 130 of the stent 100 in the state where the diameter of the stent decreases as shown in FIG. 3B.

In addition, the grooves in which the second cross sections 132 are disposed are formed to have a shape tapered toward a center O inward in the radial direction R. For this reason, when the stent 100 enters the state where the diameter of the stent decreases, the second cross sections 132 are accommodated toward the center O inward in the radial direction R along the shapes of the grooves.

The side surfaces of the first cross sections 131 and the second cross sections 132 that face each other in the circumferential direction D2 are partially in contact with each other. Accordingly, it is possible to maintain the second cross sections 132 accommodated between the plurality of adjacent first cross sections 131 in the circumferential direction D2 without positional displacement in the circumferential direction D2.

Furthermore, the apexes 132a positioned on the inner peripheral surface side of the second cross sections 132 abut on contact points P of the apexes 131a and 131b of the plurality of first cross sections 131 adjacent to each other in the circumferential direction D2. For this reason, the second cross sections 132 can be reliably disposed between the plurality of first cross sections 131 adjacent to each other in the circumferential direction D2 without positional displacement in the circumferential direction D2. Moreover, the apexes 132a of the second cross sections 132 can be prevented from abutting on the outer surface of the balloon 200 and to prevent any damage on the balloon 200. In addition, as will be described below, when the diameter of the plurality of strut cross-sectional portions 130 increases outward in the radial direction R in accordance with the dilation of the balloon 200, and therefore, the first cross sections 131 can reliably guide the increase in the diameter of the second cross sections 132 outward in the radial direction R.

The second cross sections 132 function to secure the expansion force of the stent 100 when the stent 100 enters the state where the diameter of the stent increases, instead of the function of defining the decreasable diameter of the stent 100 which is served by the first cross sections 131.

Hereinafter, a process until the stent 100 of the present embodiment enters the state where the diameter of the stent increases will be described.

First, when the balloon 200 is dilated, the diameter of the first cross sections 131 increases outward in the radial direction R due to the dilation force of the balloon 200 transmitted to the first cross sections 131. Next, the side surfaces 151 of the first cross section 131 facing the second cross sections 132 in the circumferential direction D2 transmits the dilation force of the balloon 200 to adjacent second cross sections 132 in accordance with the increase in the diameter of the first cross sections 131 outward in the radial direction R. That is, the increase in the diameter of the second cross sections 132 outward in the radial direction R is guided using the force of increasing the diameter of the side surfaces 151 of the first cross sections 131 being in contact with both sides in the circumferential direction D2. For this reason, the dilation force of the balloon 200 is transmitted to the second cross sections 132 through the first cross sections 131. Accordingly, the dilation force of the balloon 200 is reliably transmitted to both of the first cross sections 131 and the second cross sections 132. Therefore, it is possible to uniformly make the stent 100 enter the state where the diameter of the stent increases.

At this time, the apexes 132a positioned on the inner peripheral surface side of the second cross sections 132 abut on the contact points P of the apexes 131a and 131b of the plurality of first cross sections 131 adjacent to each other in the circumferential direction D2. Therefore, the force of increasing the diameter of the first cross sections 131 outward in the radial direction R is reliably transmitted to the second cross sections 132 while having the contact points P as starting points in accordance with the dilation of the balloon 200. Thus, it is possible to more uniformly increase the diameter of the plurality of strut cross-sectional portions 130 formed of the two shapes.

Note that the first cross sections 131 can more easily transmit the dilation force of the balloon 200 to the second cross sections 132 by providing the guiding portion, which guides the increase in the diameter of the second cross sections 132 outward in the radial direction R, on the side surfaces 150 of the first cross sections 131 facing the second cross sections 132 in the circumferential direction D2. As a result, it is possible to make the stent 100 reliably enter the state where the diameter of the stent increases.

In accordance with an exemplary embodiment, It can be desirable to form the struts 111 constituting the plurality of strut cross-sectional portions 130 to have at least two cross-sectional shapes in order to maintain the expansion force of the stent 100 while decreasing the decreasable diameter of the stent 100 as the stent 100 of the present embodiment. Even if it is assumed that the plurality of strut cross-sectional portions 130 have one cross-sectional shape exhibiting the same effect as that of the present embodiment, a substantially trapezoidal shape of which the upper base is longer than the lower base can be considered as the cross-sectional shape of each of the struts 111 constituting the plurality of strut cross-sectional portions 130.

However, in a case where the cross-sectional shapes of the struts 111 are made to be the above-described substantially trapezoidal shapes, the cross-sectional area of one strut 111 becomes large. Accordingly, when the stent 100 is made to enter the state where the diameter of the stent increases, the occupying ratio of the area of the struts 111 to the area of the inner peripheral surface of the body lumen wall becomes larger compared to the same ratio in the case of the strut cross-sectional portions 130 formed of at least two cross-sectional shapes. For this reason, in the case of the plurality of strut cross-sectional portions 130 formed of two cross-sectional shapes, it is possible to decrease a load on the body lumen due to faster formation of neointima after the stent 100 is indwelled, compared to the plurality of strut cross-sectional portions 130 formed of only the substantially trapezoidal cross-sectional shape.

Next, a value (ratio) obtained by dividing the decreasable diameter of the stent by the sum (total area) of the areas of the plurality of strut cross-sectional portions is set to P, and values P will be described with reference to Tables 1 to 3 while comparing the values P between a conventional example, the present embodiment, and the comparative example. In Tables 1 to 3, the value P according to the conventional example is denoted as a reference value P1, the value P according to the present embodiment is denoted as P2, and the value P according to the comparative example is denoted as P3. Note that the stent 100 exemplified as the conventional example is formed such that the cross-sectional shapes of the struts 111 are only rectangular.

The decreasable diameter of each stent 100 in the conventional example and the present embodiment is a value obtained by adding a value, in which the length H (wall thickness) of each strut 111 in the thickness direction D3 is doubled, to the diameter L of an inscribed circle of a regular polygon having the length t (line width) of each of the struts 111 in the width direction W constituting the inner peripheral surface of the stent 100 in a state where the diameter of the stent 100 decreases, as one side. That is, the decreasable diameter of the stent 100 can be expressed by Formula 1.

Decreasable diameter of stent 100=L+2H Formula (1)

The decreasable diameter of the stent 300 of the comparative example is a diameter L of an inscribed circle of a regular polygon having the length t (line width) of each of the struts 111 in the width direction W constituting the outer peripheral surface of the stent 300 in a state where the diameter of the stent 300 decreases, as one side. That is, the decreasable diameter of the stent 300 can be expressed by Formula 2.

Decreasable dimension of stent 300=L Formula (2)

Note that the radius r of a regular n-polygon having the length t of each of the struts 111 in the width direction W constituting the inner peripheral surface or the outer peripheral surface of the stent, as one side can be expressed by Formula 3. Therefore, the diameter L of the inscribed circle can be expressed by Formula 4.

r=t/2 tan(π/n) Formula (3)

L=2r=t/tan(π/n) Formula (4)

Note that the wall thickness H (the length in the thickness direction D3) and the line width t (the length in the width direction W) of each of the struts 111 constituting the cross section perpendicular to the axial direction D1 of the struts 110 of the cylindrical shape were respectively set to 80 μm and 90 μm. The sum of the areas of the plurality of strut cross-sectional portions according to each of the conventional example, the present embodiment, and the comparative example was calculated using the wall thickness H and the line width t of the above-described struts 111 in accordance with the cross-sectional shapes of the struts 111. In Tables 1 to 3, the value obtained by dividing the decreasable diameter obtained in the above by the sum of the areas of the plurality of strut cross-sectional portions is set to P.

Note that the reference values P1 in Tables 1 to 3 are respectively set to be P1=4.00, P1=5.00, and P1=6.00.

In addition, the values P corresponding to patterns in which the number of struts 111 constituting the cross section in a direction perpendicular to the axial direction D1 of the struts 110 of the cylindrical shape is 12, 16, and 20 are denoted when the values P2 and P3 are composed of the patterns.

In addition, a change rate z1 and a change rate z2 of P2 and P3 with respect to the reference value P1 are denoted in order to compare P2 with P3 in Tables 1 to 3. Note that the change rate z1 and the change rate z2 can be respectively expressed by Formula (5) and Formula (6).

z1=(P2−P1)/P1×100 Formula (5)

z2=(P3−P1)/P1×100 Formula (6)

TABLE 1
Table 1: P2, P3, change rate z1, and change
rate z2 in each of number of struts

| Reference value P1 = 4.00 | Number of struts | | |
|---|---|---|---|
| Cross-sectional shape | 12 | 16 | 20 |
| Present embodiment P2 | 5.28 | 4.97 | 4.80 |
| Comparative example | 7.73 | 7.85 | 7.94 |
| (triangular shape) P3 | | | |
| Change rate z1 (present embodiment) | 32.0 | 24.2 | 20.0 |
| Change rate z2 (comparative example) | 93.3 | 96.2 | 98.6 |

TABLE 2
Table 2: P2, P3, change rate z1, and change
rate z2 in each of number of struts

| Reference value P1 = 5.00 | Number of struts | | |
|---|---|---|---|
| Cross-sectional shape | 12 | 16 | 20 |
| Present embodiment P2 | 5.28 | 4.97 | 4.80 |
| Comparative example | 7.73 | 7.85 | 7.94 |
| (triangular shape) P3 | | | |
| Change rate z1 (present embodiment) | 5.64 | −0.66 | −3.97 |
| Change rate z2 (comparative example) | 54.6 | 56.9 | 58.9 |

TABLE 3
Table 3: P2, P3, change rate z1, and change
rate z2 in each of number of struts

| Reference value P1 = 6.00 | Number of struts | | |
|---|---|---|---|
| Cross-sectional shape | 12 | 16 | 20 |
| Present embodiment P2 | 5.28 | 4.97 | 4.80 |
| Comparative example | 7.73 | 7.85 | 7.94 |
| (triangular shape) P3 | | | |
| Change rate z1 (present embodiment) | −12.0 | −17.2 | −20.0 |
| Change rate z2 (comparative example) | 28.9 | 30.8 | 32.4 |

As shown in Tables 1, 2, and 3, the change rate z1 of P2 of the present embodiment when the reference value P1 is within the range of 4.00 to 6.00 is shifted within the range of −20.0% to 32.0%.

In accordance with an exemplary embodiment, the change rate z2 of the comparative example when the reference value P1 is within the range of 4.00 to 6.00 is shifted within the range of 28.9% to 98.6%.

The variation of the change rate z2 of P3 of the comparative example is larger than that of the change rate z1 of P2 of the present embodiment. It is considered that this is because the change rate z2 of P3 with respect to the reference value P1 in the comparative example increases since a decrease value of the total area of the plurality of strut cross-sectional portions 330 is larger than that of the decreasable diameter by making the strut cross-sectional portions 330 have a triangular shape.

In accordance with an exemplary embodiment, the plurality of strut cross-sectional portions 130 of the present embodiment have the first cross sections 131 for decreasing the decreasable diameter and the second cross sections 132 for securing the expansion force of the stent 100 when the diameter of the stent 100 increases. Therefore, the decrease value of the total area of the plurality of strut cross-sectional portions 130 with respect to the decrease value of the decreasable diameter of the stent 100 can be further suppressed compared to the comparative example. For this reason, it is possible to suppress the variation of the change rate z1 of P2 with respect to the reference value P1 to be small compared to the comparative example.

Accordingly, in the stent 100 of the present embodiment, the change rate z1 is preferably in the range of −20.0% to 28.8% and more preferably −20.0% to 28.0%. When the change rate z1 of the stent 100 falls within these ranges, it is possible to decrease the decreasable diameter of the stent 100 and to sufficiently secure the total area of the plurality of strut cross-sectional portions 130. Therefore, it is possible to improve the passing properties and the expansibility of the stent 100.

As described above, in the stent 100 of the present embodiment, the stent 100 has a plurality of linear struts 110, which form an outer periphery of a cylindrical shape and in which a gap is formed, and a link portion 120 connecting the plurality of linear struts 110 to each other in the gap, and in which the struts 110 of the cylindrical shape are mounted on an outer peripheral surface of a dilatable and deformable balloon 200 in a state where the diameter of the struts decreases, in which when the struts 110 are mounted on the outer peripheral surface of the balloon 200 which is in a deflated state, in the state where the diameter of the struts decreases, the stent 100 has a plurality of strut cross-sectional portions 130 in a cross section perpendicular to an axial direction of the struts 110 of the cylindrical shape, the strut cross-sectional portions 130 adjacent to each other in a circumferential direction of the struts 110 of the cylindrical shape have shapes different from each other, and surfaces of the strut cross-sectional portions 130 adjacent to each other in the circumferential direction are in contact with each other when the struts 110 are mounted on the outer peripheral surface of the balloon 200, which is in the deflated state, in the state where the diameter of the struts decreases.

According to the stent 100 having the above-described configuration, it is possible to decrease the decreasable diameter of the stent 100 while securing the cross-sectional area of the struts 111. Accordingly, when delivering the stent 100, it is possible to deliver the stent 100 in a state where the decreasable diameter of the stent 100 is made to be small. Therefore, the passing properties of the stent 100 can be improved. Furthermore, after the stent 100 is delivered, it is possible to secure the expansion force of the stent 100 when the stent 100 is indwelled in a lesion site in the state where the diameter of the stent increases.

In addition, the plurality of strut cross-sectional portions 130 are provided in the cross section perpendicular to the axial direction D1 of the cylindrical shape at both end portions of the struts 110 of the cylindrical shape. Accordingly, in the stent 100, it is possible to decrease the decreasable diameter at both end portions and to suppress the clearance caused by the difference in the outer diameter at both end portions of the stent 100 and the balloon 200 during the delivery of the stent 100. For this reason, it is possible to prevent the shape of the stent 100 from being unintentionally changed or the stent 100 from dropping out due to the stent 100 being caught in a bent site, a stenosed site, or the like during the delivery of the stent 100. Therefore, the passing properties of the stent 100 can be further improved.

In addition, the plurality of strut cross-sectional portions 130 have the first cross sections 131 and the second cross sections 132 which are adjacent to each other along the circumferential direction D2 of the cylindrical shape and formed of shapes different from each other, and the first cross sections 131 and the second cross sections 132 are alternately arranged along the circumferential direction of the cylindrical shape. Accordingly, the first cross sections 131 and the second cross sections 132 constituting the plurality of strut cross-sectional portions 130 are arranged along the circumferential direction D2 in a state of being in close proximity to each other. Therefore, it is possible to decrease the decreasable diameter of the stent 100.

In addition, the length t1 of the inner peripheral surface of a first cross section in a width direction W is longer than the length t2 of an inner peripheral surface of a second cross section in the width direction W when the struts 110 of the cylindrical shape are mounted on the outer peripheral surface of the balloon 200 and enter the state where the diameter of the struts decreases. Accordingly, the first cross section can secure a sufficient contact area with respect to the outer surface of the balloon 200. For this reason, the dilation force of the balloon 200 can be reliably transmitted to the first cross section 131, and therefore, a failure of expansion of the stent 100 can be prevented.

In addition, the first cross section 131 and the second cross section 132 are formed such that profiles of the respective cross sections are in an offset relation in the thickness direction, and are arranged in close proximity to each other in the circumferential direction D2 of the cylindrical shape in the state where the diameter of the struts 110 of the cylindrical shape, which are mounted on an outer surface of the balloon 200, decreases. Accordingly, the first cross section 131 and the second cross section 132 enters a state where the first cross section and the second cross section are in closer proximity to each other, and therefore, it is possible to further decrease the decreasable diameter of the stent 100.

In addition, the first cross section 131 has a guiding portion, which guides expansion of the second cross section 132 during dilation of the balloon 200, on at least a part of a side surface positioned on a side opposite to the second cross section 132 in the circumferential direction D2 of the struts 110 of the cylindrical shape, and transmits a dilation force of the balloon 200 to the second cross section 132. Accordingly, the first cross section 131 further facilitates the transmission of the dilation force of the balloon 200 to the second cross section 132. For this reason, the dilation force of the balloon 200 is reliably transmitted to both of the first cross section 131 and the second cross section 132. Therefore, it is possible to make the stent 100 enter the state where the diameter of the stent uniformly increases.

### Modification Example 1 of Strut Cross-Section

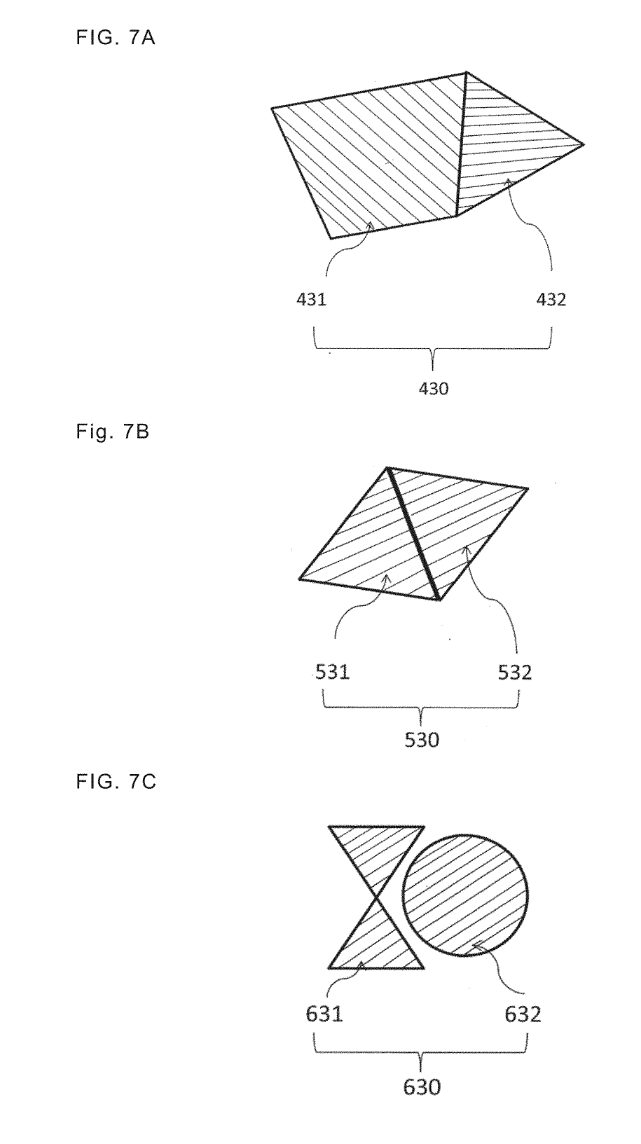

In the above-described embodiment, the first cross sections 131 and the second cross sections 132 constituting the plurality of stent cross-sectional portions 130 respectively have the rectangular shape and the inverted triangular shape. However, the shapes of the first cross sections 131 and the second cross sections 132 are not particularly limited as long as the first cross sections and the second cross sections are formed such that profiles of the respective cross sections are in an offset relation in the thickness direction. For example, as shown in FIG. 7A, a plurality of strut cross-sectional portions 430 may have a combined shape such that a first cross section 431 has a trapezoidal shape of which the upper base is longer than the lower base and a second cross section 432 has an inverted triangular shape.

### Modification Example 2 of Strut Cross-Section

In the above-described embodiment, the first cross sections 131 and the second cross sections 132 constituting the plurality of stent cross-sectional portions 130 respectively have the rectangular shape and the inverted triangular shape. However, the shapes of the first cross sections 131 and the second cross sections 132 are not particularly limited as long as the first cross sections and the second cross sections are formed such that profiles of the respective cross sections are in an offset relation in the thickness direction. For example, as shown in FIG. 7B, a plurality of strut cross-sectional portions 530 may have a combined shape such that a first cross section 531 has a triangular shape and a second cross section 532 has an inverted triangular shape.

### Modification Example 3 of Strut Cross-Section

In the above-described embodiment, the first cross sections 131 and the second cross sections 132 constituting the plurality of stent cross-sectional portions 130 respectively have the rectangular shape and the inverted triangular shape. However, the shapes of the first cross sections 131 and the second cross sections 132 are not particularly limited as long as the first cross sections and the second cross sections are formed such that profiles of the respective cross sections are in an offset relation in the thickness direction. For example, as shown in FIG. 7C, a plurality of strut cross-sectional portions 630 may have a combined shape such that a first cross section 631 has a shape obtained by interlocking two triangular shapes, of which the upper direction and the lower direction are reversed with each other, at apexes and a second cross section 631 has a circular shape.

The present disclosure is not limited to the embodiment and the modification examples described above, and can be variously modified within the claims.

In addition, the forms of the struts are not also limited to the embodiment and the modification examples described above. The stent of the present disclosure may not be formed of struts, which extend in the circumferential direction D2 around the axial direction D1 while being folded back in a wave shape and form an endless annular shape, like the struts 110 of the above-described embodiment. However, the struts 110 may be formed such that a strut forming an annular shape is provided at both end portions and extends in a spiral shape around the axial direction D1 while being folded in a wave shape between a strut at one end and a strut at the other end.

In addition, the present disclosure includes a form which does not contain a coating body and a form in which a medicine capable of suppressing proliferation of neointima is included in a biodegradable material. In the latter form, the medicine is gradually eluted in accordance with the decomposition of the biodegradable material, and restenosis of a lesion site is suppressed.

The detailed description above describes a stent used for treating, for example, a stenosed site or a closed site caused in a body lumen such as a blood vessel. The invention is not limited, however, to the precise embodiments and variations described. Various changes, modifications, and equivalents can be effected by one skilled in the art without departing from the spirit and scope of the invention as defined in the accompanying claims. It is expressly intended that all such changes, modifications, and equivalents which fall within the scope of the claims are embraced by the claims.

What is claimed is:

1. A stent comprising:
a plurality of linear struts, which form an outer periphery of a cylindrical shape in which a gap is formed;
a link portion connecting the plurality of linear struts to each other in the gap, wherein the plurality of linear struts of the cylindrical shape are mounted on an outer peripheral surface of a dilatable and deformable balloon in a state where a diameter of the plurality of linear struts decreases; and
wherein the stent has a plurality of strut cross-sectional portions in a cross section perpendicular to an axial direction of the plurality of linear struts of the cylindrical shape, the plurality of strut cross-sectional portions adjacent to each other in a circumferential direction of the plurality of linear struts of the cylindrical shape have shapes different from each other, and surfaces of the plurality of strut cross-sectional portions are in contact with each other when the plurality of linear struts are mounted on the outer peripheral surface of the balloon, which is in a deflated state, in a state where the diameter of the plurality of linear struts decreases.

2. The stent according to claim 1, wherein the plurality of strut cross-sectional portions are provided on the cross section perpendicular to the axial direction of the struts of the cylindrical shape at both end portions of the plurality of linear struts of the cylindrical shape.

3. The stent according to claim 1, wherein the plurality of strut cross-sectional portions are adjacent to each other along the circumferential direction of the cylindrical shape and have a first cross section and a second cross section which have shapes different from each other and are alternately arranged along the circumferential direction of the cylindrical shape.

4. The stent according to claim 3, wherein a length of an inner peripheral surface of the first cross section in a width direction is longer than that of an inner peripheral surface of the second cross section in the width direction when the plurality of linear struts of the cylindrical shape are mounted on the outer peripheral surface of the balloon and to be in a state where the diameter of the struts decreases.

5. The stent according to claim 3, wherein the first cross section and the second cross section are formed such that profiles of the respective cross sections are in an offset relation in a thickness direction, and are arranged in close proximity to each other in the circumferential direction of the cylindrical shape in the state where the struts of the cylindrical shape are mounted on an outer surface of the balloon, and the diameter of the struts of the cylindrical shape decreases.

6. The stent according to claim 3, wherein the first cross section has a rectangular shape and the second cross section has an inverted triangular shape.

7. The stent according to claim 6, wherein the plurality of strut cross-sectional portions comprises an equal number of the first cross sections having the rectangular shape and the second cross sections have the inverted triangular shape.

8. The stent according to claim 7, wherein the equal number of the first cross sections having the rectangular shape and the second cross sections having the inverted triangular shape is eight.

9. The stent according to claim 6, wherein a width of an inner peripheral surface of the first cross section is longer than a width of an inner peripheral surface of the second cross section.

10. The stent according to claim 9, wherein a height of the first cross section is equal to a length of a side surface of the second cross section, which faces the first cross section in the circumferential direction.

11. The stent according to claim 3, where the plurality of linear struts include a plurality of straight portion and a plurality of bent portions interlocking the plurality of straight portions extending in directions different from each other, and wherein the bent portion has a hollow portion formed from an inner peripheral side toward an outer peripheral side of the plurality of linear struts at a central portion.

12. The stent according to claim 3, wherein the first cross section and the second cross section each have an inverted triangular shape.

13. The stent according to claim 3, wherein the first cross section has a trapezoidal shape and the second cross section has an inverted triangular shape.

14. The stent according to claim 3, wherein the first cross section has a triangular shape and the second cross section has an inverted triangular shape.

15. The stent according to claim 3, wherein the first cross section has a shape obtained by interlocking two triangular shapes, of which an upper direction and a lower direction are reversed with each other, at apexes and the second cross section has a circular shape.

16. A stent comprising:
a plurality of linear struts, which form an outer periphery of a cylindrical shape; and
a link portion connecting the plurality of linear struts to each other, wherein the stent has a plurality of strut cross-sectional portions in a cross section perpendicular to an axial direction of the plurality of linear struts of the cylindrical shape, and wherein the plurality of strut cross-sectional portions adjacent to each other in a circumferential direction of the plurality of linear struts of the cylindrical shape have shapes different from each other.

17. The stent according to claim 16, further comprising:
a dilatable and deformable balloon, and wherein the plurality of linear struts is mounted on an outer peripheral surface of the dilatable and deformable balloon.

18. The stent according to claim 17,
wherein surfaces of the plurality of strut cross-sectional portions are in contact with each other when the plurality of linear struts are mounted on the outer peripheral surface of the balloon, which is in a deflated state, in a state where the diameter of the plurality of linear struts decreases; and
wherein the plurality of strut cross-sectional portions are provided on the cross section perpendicular to the axial direction of the struts of the cylindrical shape at both end portions of the struts of the cylindrical shape.

19. The stent according to claim 18, wherein the plurality of strut cross-sectional portions has a first cross section and a second cross section which have shapes different from each other and are alternately arranged along the circumferential direction of the cylindrical shape, and wherein the first cross section has a rectangular shape and the second cross section has an inverted triangular shape.

20. A method of decreasing an outer diameter of a stent in a deflated state, the method comprising:
forming an outer periphery of a cylindrical shape with a plurality of linear struts having a gap between the plurality of linear struts;
connecting the plurality of linear struts to each other in the gap with a link portion; and
mounting the plurality of linear struts of the cylindrical shape on an outer peripheral surface of a dilatable and deformable balloon, wherein the stent has a plurality of strut cross-sectional portions in a cross section perpendicular to an axial direction of the plurality of linear struts of the cylindrical shape, the wherein the plurality of strut cross-sectional portions adjacent to each other in a circumferential direction of the plurality of linear struts of the cylindrical shape have shapes different from each other, and surfaces of the strut cross-sectional portions are in contact with each other when the plurality of linear struts are mounted on the outer peripheral surface of the balloon.
